(19) United States
(12) Patent Application Publication
(10) Pub. No.: US 2017/0080040 A1
(43) Pub. Date: Mar. 23, 2017

(54) DECOCTION OF OLIVE LEAVES

(71) Applicant: Livio Pesle, San Giovanni al Natisone (IT)

(72) Inventor: Livio Pesle, San Giovanni al Natisone (IT)

(21) Appl. No.: 15/311,470

(22) PCT Filed: May 8, 2015
(86) PCT No.: PCT/IB2015/053381

§ 371 Date: Nov. 15, 2016

(30) Foreign Application Priority Data
May 15, 2014 (IT) UD2014A000080

(51) Int. Cl. A61K 36/63; A61K 8/97; A61K 31/7048; A61K 8/60; A61K 31/05; A61K 8/34; A61K 31/351; A61K 8/49; A61K 31/7016; C11D 3/382; C11D 3/20; C11D 3/22; A23L 2/52; A23L 33/105; A23K 20/10; A61Q 15/00

(52) U.S. Cl. CPC A61K 36/63; A61Q 15/00; A61K 8/97; A61K 31/7048; A61K 8/60; A61K 31/05; A61K 8/347; A61K 31/351; A61K 8/498; A61K 31/7016; C11D 3/382; C11D 3/2096; C11D 3/2065; C11D 3/2058; C11D 3/222; A23L 2/52; A23L 33/105; A23K 20/10; A61K 2236/00; A23V 2002/00

(57) ABSTRACT

A decoction of olive leaves comprises an aqueous composition containing at least from about 500 mg/liter to about 3,000 mg/liter of oleuropein, from about 100 mg/liter to about 300 mg/liter of hydroxytyrosol, from about 90 mg/liter to about 280 mg/liter of tyrosol, from about 400 mg/liter to about 1,800 mg/liter of elenolic acid.

### FIELD OF THE INVENTION

The present invention concerns a decoction of the leaves of the olive tree with a taste profile that can be appreciated by consumers and with high nutritional and/or nutraceutical properties.

### BACKGROUND OF THE INVENTION

Olea europea L., commonly called olive, is an evergreen arboreal plant belonging to the Oleaceae family and found in temperate zones.

Olive leaves contain different types of secondary metabolites, the concentration of which can vary considerably depending on a number of factors, including the variety, the harvesting period, the development of the branches, climatic conditions and the preservation conditions (El and Karakaya, 2009), as well as the type of cultivar (variety) of the plant itself or the organoleptic characteristics of the corresponding leaves. The main chemical constituents are represented by secoiridoids, phenolic derivatives, flavonoids, triterpenes, sterols and other lipids. Among the secoiridoids, the most abundant is oleuropein: this is the ester of the phenolic hydroxytyrosol derivative with elenolic β-glucoside acid, whose concentration in olive leaves can vary from about 1% to 14%. The other secoiridoids are represented by a series of glycosides, including ligstroside, oleuroside and oleoside, the glucoside of elenolic acid, and their similars without the sugar portion (aglycones), such as elenolic acid and aglycone of oleuropein (El and Karakaya, 2009; Laguerre et al., 2009; Fu et al., 2010; Syed, 2010 2010). The following are the formulas of the main secoiridoids of the leaves of O. europaea:

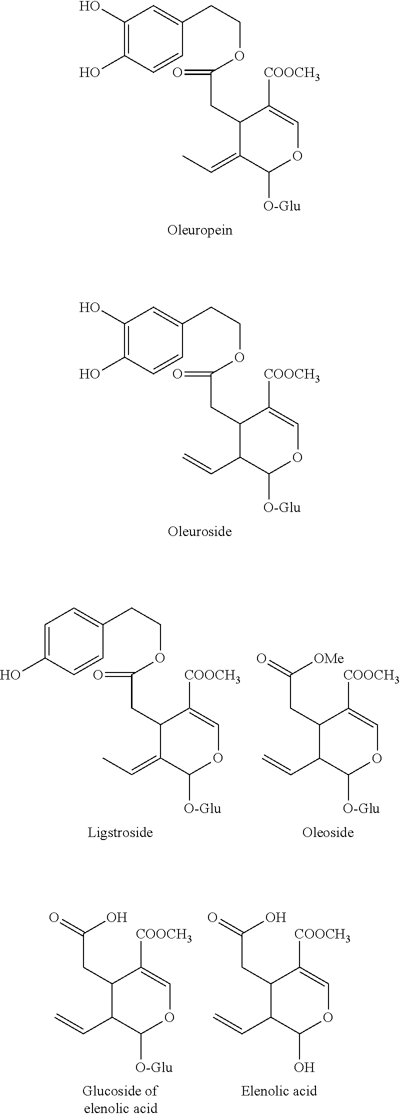

The main phenolic derivatives are the phenyl propanoids hydroxytyrosol (or 3,4-dihydroxyphenyl ethanol), tyrosol, p-coumaric acid, caffeic acid, ferulic acid and verbascoside, as well as a series of benzoic acids and derivatives, such as p-hydroxybenzoic acid, protocatechuic acid, gallic acid, vanillic acid, vanillin and syringic acid (FIG. 2) (Ryan and Robards, 1998; Paiva-Martin and Pinto, 2008; Fu et al., 2010; Korukluoglu et al., 2010; Omar, 2010; Ortega-Garcia and Peragòn, 2010). The following are the formulas of the main phenolic derivatives of the leaves of O. europaea:

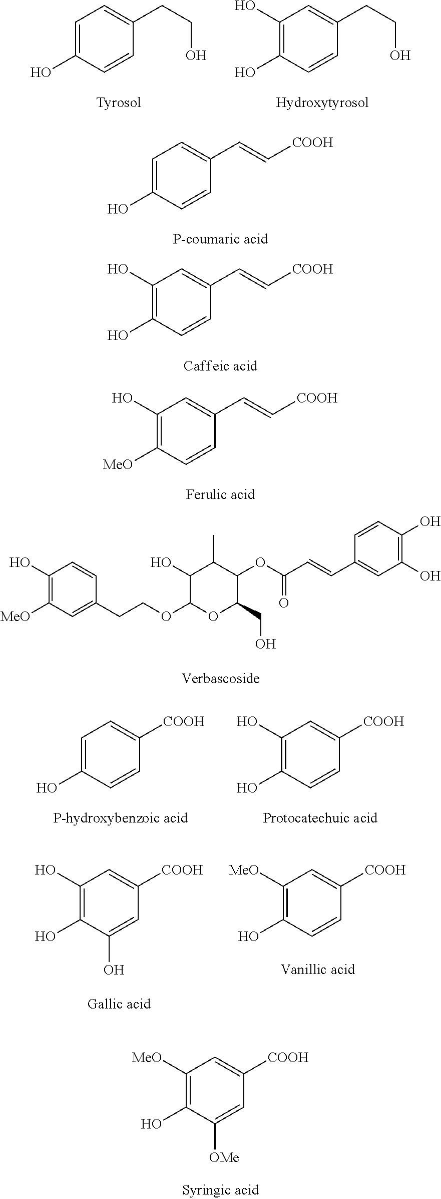

Olive leaves contain a series of flavonoids, present as aglycones (including quercetin, apigenin, luteolin, diosmetin) or as glycosides (mainly quercetin-3-O-rutinoside, or rutin, apigenin-7-O-glucoside, luteolin 7-O-glucoside, luteolin 5-O-glucoside 4). The content in total flavonoids is estimated at 858 mg of equivalents of catechin for 100 g of leaves (Ryan and Robards, 1998; El and Karakaya, 2009; Laguerre et al., 2009). The following are the formulas of the main flavonoids of the leaves of O. europaea:

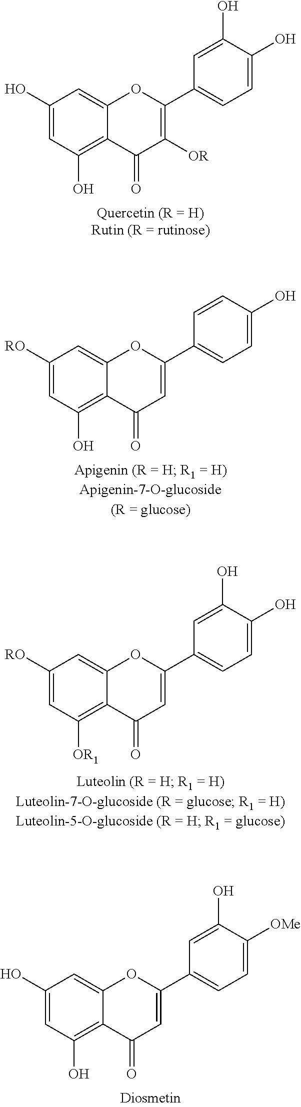

The use of olive leaves in traditional medicine is one of the most ancient legacies handed down over the centuries in populations of areas of the Mediterranean basin with a temperate climate. The leaves are known for the preparation of decoctions used as mouthwashes and, after being taken orally, in treating gastrointestinal disturbances, infections of the respiratory and urinary tracts, in controlling high blood pressure, hypercholesterolemia, hyperglycemia, and also for their diuretic properties (Khan et al., 2007). In traditional Italian medicine they are used above all for their vasodilator capacities in treating high blood pressure, but the validity of their use in this sense is not supported by random clinical studies to confirm their efficacy. The German Commission E, although it cited a series of properties attributed to olive leaves, including hypotensive, coronary dilating, antiarrhythmic, hypoglycemizing and diuretic properties, did not justify their use as a hypertensive since its clinical efficacy is not sufficiently documented. However, numerous experimental studies in vivo and/or in vitro have shown interesting biological properties for some preparations and/or for some chemical constituents of the olive leaves.

It is known that an extract of olive leaves can be useful in the treatment of stage 1 arterial hypertension.

Moreover, some biological properties of preparations with an olive leaf base are: anti-oxidizing and radical scavenger activity, protective activity on the level of the cardiovascular system, mainly due to its anti-oxidizing capacity, hypoglycemizing and hypolipidemizing activity, anti-inflammatory activity, analgesic activity, antimicrobial and antiviral activity, anti-tumoral activity, photoprotective and cicatrizant activity, the activity on the level of the bone tissue cells.

The results of many experimental studies in animals and in vitro studies support the beneficial properties attributed to olive leaves in the alleviation of different affections including high blood pressure, cardiovascular diseases, diabetes and hyperlipidemia. The beneficial effects may derive from the presence of compounds with an antioxidant activity in the leaves, above all of oleuropein and hydroxytyrosol, as well as tyrosol and elenolic acid.

It is known that decoctions of olive leaves, whether they are prepared with fresh or dried leaves, are considered a natural remedy which has been used in traditional medicine for a long time. However, these traditional decoctions do not provide an adequate contribution in terms of beneficial compounds, such as oleuropein, hydroxytyrosol, tyrosol and elenolic acid.

On this point, the article “Quantitation of Oleuropein and Related Metabolites in decoctions of Olea europaes Leaves from Ten Greek Cultivated Varieties by HPLC with Diode Array Detection (HPLC-DAD)”, Apostolis et al., Journal of Liquid Chromatography & Related Technologies®, 28: 1557-1571, 2005 reports the analysis of decoctions of olive leaves prepared from 10 different varieties of Greek olive flora (cultivars) and the quantification of the bioactive constituents oleuropein (OE), elenolic acid (EA), hydroxytyrosol (HT) and tyrosol (T).

The decoctions analyzed in Apostolis et al. were prepared according to the traditional Greek recipe, which provides to boil the olive leaves (50 g) in water (250 ml) for a short period, that is, about three minutes. These proportions are those considered standard in traditional decoctions of olive leaves.

The hot mixture is then filtered through a paper filter and the filtrate is then extracted once with EtOAc (250 ml). A portion of the EtOAc layer (50 mL) was obtained and evaporated dry at a reduced pressure to give a solid residue.

The quantification cited in Apostolis et al. indicates that:
OE Oleuropein varies from 11.8 to 121.9 mg for 250 ml of water (that is, from 47.2 to 487.6 mg per liter);
HT Hydroxytyrosol varies from 0.6 to 23.9 mg per 250 ml of water (that is, from 2.4 to 95.6 mg per liter);
T Tyrosol varies from 0.4 to 20.5 mg per 250 ml of water (that is from 1.6 to 82 mg per liter);
EA Elenolic acid varies from 0.0 to 33.1 mg per 250 ml of water (that is from 0 to 132.4 mg per liter).

Consequently, the known traditional decoctions of olive leaves generally have a reduced nutritional and nutraceutical content of the above mentioned beneficial active principles and compounds.

It is important to underline how in Apostolis et al., the compounds HT, T and EA are metabolites of OE, that is, they are not present as such but are a hydrolysis product from OE. Therefore the quantity of such compounds present in the decoction is correlated, for example proportional, to the initial quantity of OE present in the decoction, which varies considerably according to the type of olive leaves used and the method of extraction. In any case, the quantity of such compounds can be considered as produced to the detriment of the initial quantity of OE.

In general, moreover, from the point of view of taste, the traditional decoctions in question have a marked bitter taste. The compound OE, in particular, is responsible for the very bitter taste of these decoctions. Consequently, in the state of the art, it is maintained that the presence of more or less high quantities of OE influences the taste of these decoctions, making them more or less bitter. Therefore, since these known decoctions are often bitter, they can also be less appreciated by consumers.

Documents WO-A-2004/0389962, EP-A-1.582.512 and EP-A-0.937.455 are also known, which describe extracts of olive leaves.

Moreover, the following scientific articles are known, which describe uses of olive leaves and their extracts: Sedef et al. “Olive tree (Olea europaea) leaves: potential beneficial effects on human health” Nutrition Reviews, vol. 67, no. 11; Stavros et al. “Enrichment of table olives with polyphenols extracted from olive leaves”, Food Chemistry, Elsevier Ltd, NL, vol. 127, no. 4 and Syed Haris Omar “Oleuropein in Olive and its Pharmacological Effects”, Scientia Pharmaceutica, vol. 78, no. 2.

There is therefore a need to make available a decoction of olive leaves that can overcome at least one of the disadvantages of the state of the art.

The Applicant has devised, tested and embodied the present invention to overcome the shortcomings of the state of the art and to obtain these and other purposes and advantages.

Unless otherwise defined, all the technical and scientific terms used here and hereafter have the same meaning as commonly understood by a person with ordinary experience in the field of the art to which the present invention belongs. Even if methods and materials similar or equivalent to those described here can be used in practice and in the trials of the present invention, the methods and materials are described hereafter as an example. In the event of conflict, the present application shall prevail, including its definitions. The materials, methods and examples have a purely illustrative purpose and shall not be understood restrictively.

The word “comprise” and variants of the word such as “comprises” and “comprising” are used here to indicate the inclusion of a clearly expressed whole or clearly expressed wholes but not the exclusion of any other whole or any other wholes, unless in the context or in use an exclusive interpretation of the word is required.

### SUMMARY OF THE INVENTION

The present invention is set forth and characterized in the independent claims, while the dependent claims describe other characteristics of the invention or variants to the main inventive idea.

In accordance with the above purposes, forms of embodiment described here concern a decoction of olive leaves that overcomes the limits of the state of the art and eliminates the defects present therein.

According to one form of embodiment, the decoction of olive leaves (Olea europaea L.) comprises an aqueous composition containing at least:

oleuropein (OE) from about 500 mg/liter to about 3000 mg/liter;

hydroxytyrosol (HT) from about 100 mg/liter to about 300 mg/liter;

tyrosol (T) from about 90 mg/liter to about 280 mg/liter;

elenolic acid (EA) from about 400 mg/liter to about 1800 mg/liter.

According to another form of embodiment, the decoction can also include rutin between about 100 mg/liter and about 600 mg/liter.

In accordance with possible forms of embodiment, said decoction is an extract of said olive leaves obtained by extraction with hot water.

These and other aspects, characteristics and advantages of the present disclosure will be better understood with reference to the following description, drawings and attached claims. The drawings, which are integrated and form part of the present description, show some forms of embodiment of the present invention, and together with the description, are intended to describe the principles of the disclosure.

The various aspects and characteristics described in the present description can be applied individually where possible. These individual aspects, for example aspects and characteristics described in the attached dependent claims, can be the object of divisional applications.

It is understood that any aspect or characteristic that is discovered, during the patenting process, to be already known, shall not be claimed and shall be the object of a disclaimer.

### BRIEF DESCRIPTION OF THE DRAWING

FIG. 1 contains two graphical descriptions illustrating the effect of the decoctions and the caffeine on the latency between the ethanol injection and the loss of the straightening reflex and on the duration of the latter.

### DETAILED DESCRIPTION OF FORMS OF EMBODIMENT

We shall now refer in detail to the various forms of embodiment of the present invention, of which one or more examples are shown in the attached drawing. Each example is supplied by way of illustration of the invention and shall not be understood as a limitation thereof. For example, the characteristics shown or described insomuch as they are part of one form of embodiment can be adopted on, or in association with, other forms of embodiment to produce another form of embodiment. It is understood that the present invention shall include all such modifications and variants.

Before describing these forms of embodiment, we must also clarify that the present description is not limited in its application to details of the construction and disposition of the components as described in the following description using the attached drawings. The present description can provide other forms of embodiment and can be obtained or executed in various other ways. We must also clarify that the phraseology and terminology used here is for the purposes of description only, and cannot be considered as limitative.

All the intervals reported here shall be understood to include the extremes, including those that report an interval “between” two values, unless otherwise indicated.

The present description also includes the intervals that derive from uniting or overlapping two or more intervals described, unless otherwise indicated.

The present description also includes the intervals that can derive from the combination of two or more values taken at different points, unless otherwise indicated.

Forms of embodiment described here concern a decoction of olive leaves comprising an aqueous composition containing oleuropein (OE) from about 500 mg/liter to about 3000 mg/liter.

Other forms of embodiment described here concern a decoction of olive leaves comprising an aqueous composition containing oleuropein (OE) from about 600 mg/liter to about 3000 mg/liter.

Other forms of embodiment described here concern a decoction of olive leaves comprising an aqueous composition containing oleuropein (OE) from about 700 mg/liter to about 3000 mg/liter.

Other forms of embodiment described here concern a decoction of olive leaves comprising an aqueous composition containing oleuropein (OE) from about 800 mg/liter to about 3000 mg/liter.

Yet other forms of embodiment described here concern a decoction of olive leaves comprising an aqueous composition containing oleuropein (OE) from about 1000 mg/liter to about 3000 mg/liter.

Other forms of embodiment described here concern a decoction of olive leaves comprising an aqueous composition containing oleuropein (OE) from about 1200 mg/liter to about 3000 mg/liter.

Forms of embodiment described here concern a decoction of olive leaves comprising an aqueous composition containing hydroxytyrosol (HT) from about 100 mg/liter to about 300 mg/liter.

Forms of embodiment described here concern a decoction of olive leaves comprising an aqueous composition containing tyrosol (T) from about 90 mg/liter to about 280 mg/liter.

Forms of embodiment described here concern a decoction of olive leaves comprising an aqueous composition containing elenolic acid (EA) from about 400 mg/liter to about 1800 mg/liter.

Forms of embodiment described here concern a decoction of olive leaves comprising an aqueous composition containing elenolic acid (EA) from about 500 mg/liter to about 1800 mg/liter.

Forms of embodiment described here concern a decoction of olive leaves comprising an aqueous composition containing at least:

oleuropein (OE) from about 500 mg/liter to about 3000 mg/liter;

hydroxytyrosol (HT) from about 100 mg/liter to about 300 mg/liter;

tyrosol (T) from about 90 mg/liter a about 280 mg/liter;

elenolic acid (EA) from about 400 mg/liter to about 1800 mg/liter.

Other forms of embodiment described here concern a decoction of olive leaves comprising an aqueous composition containing at least:

oleuropein (OE) from about 1200 mg/liter to about 3000 mg/liter;

hydroxytyrosol (HT) from about 100 mg/liter to about 300 mg/liter;

tyrosol (T) from about 90 mg/liter to about 280 mg/liter;

elenolic acid (EA) from about 500 mg/liter to about 1800 mg/liter.

By the expression “decoction” we intend an extract with hot water, that is, an extract obtained by means of an extraction process in high temperature conditions, normally boiling temperature, of the solvent in which the olive leaves are immersed and used as extraction medium, in this case water, that is, in this specific case an extraction obtained using hot or boiling water in which the olive leaves are immersed. Clearly, the expression “extract with hot water” must not be intended as limited to a decoction or extract that is “hot” in itself, in the sense that the final decoction or extract can be used even once it has cooled.

The decoction in accordance with the present description therefore has a much higher content of each of the beneficial bioactive compounds OE, HT, T and EA than known traditional decoctions.

Applicant has also found that the compounds HT, T and EA that are present exclusively as a result of the hydrolysis of the OE compound, are not produced to the detriment of the quantity of OE compound. For example this is thanks to a particular mix of olive leaves suitably selected and exceptionally rich in OE Oleuropein. In this way, the decoction in accordance with the present description can exceptionally boast both a high quantity of OE compound and a high quantity of HT, T and EA compounds.

Moreover, Applicant has found that, most surprisingly, the decoction according to the present description does not have a bitter or very bitter taste, despite the high presence of OE. The decoction in accordance with the present description has, in fact, a slightly grassy taste with only a slightly bitter taste. Without being constrained to theory, Applicant maintains that this surprising effect on the taste of the decoction according to the present description is due to the fact that there are also present in it high quantities of the compounds HT, T and EA, higher than in the state of the art, which have a positive effect in moderating the flavor and bitter taste which would result from a high quantity of OE, rendering the decoction overall only slightly bitter and therefore much more appreciable by consumers. In particular Applicant has found that the bitter taste increases or decreases, given the same OE content, in relation to a respectively more or less high quantity of HT, T and EA in the decoctions.

In accordance with possible forms of embodiment, combinable with all the forms of embodiment described here, the decoction comprises only water as the only solvent, as well as at least the beneficial bioactive compounds as above, that is, at least OE, HT, T and EA. In other words, according to possible forms of embodiment, the decoction is a mixture in water of at least the beneficial bioactive compounds as above, that is, OE, HT, T and EA.

In accordance with possible forms of embodiment, combinable with all the forms of embodiment described here, the decoction can also include a sugar (for example glucose, fructose, saccharose, lactose, galactose, hydroxylated starch) or a sweetener or a sugar substitute or other sweetener.

In accordance with possible forms of embodiment, combinable with all the forms of embodiment described here, the oleuropein (OE) content can be comprised between about 500 mg/liter and about 2900 mg/liter, in particular between 600 mg/liter and 2850 mg/liter, more in particular between about 700 mg/liter and about 2800 mg/liter, more in particular between about 800 mg/liter and about 2750 mg/liter, even more in particular between about 900 mg/liter and about 2700 mg/liter, even more in particular between about 1000 mg/liter and about 2600 mg/liter.

In accordance with possible forms of embodiment, combinable with all the forms of embodiment described here, the oleuropein (OE) content can be comprised between about 1500 mg/liter and about 2800 mg/liter, in particular between 1600 mg/liter and 2700 mg/liter, more in particular between about 1550 mg/liter and about 2650 mg/liter, even more in particular between about 1500 mg/liter and about 2600 mg/liter.

In accordance with possible forms of embodiment, combinable with all the forms of embodiment described here, the hydroxytyrosol (HT) content can be comprised between about 110 mg/liter and about 290 mg/liter, in particular between 140 mg/liter and 280 mg/liter, more in particular between about 150 mg/liter and about 250 mg/liter, even more in particular between about 180 mg/liter and about 240 mg/liter.

In accordance with possible forms of embodiment, combinable with all the forms of embodiment described here, the tyrosol (T) content can be comprised between about 95 mg/liter and about 270 mg/liter, in particular between 110 mg/liter and 250 mg/liter, more in particular between about 125 mg/liter and about 230 mg/liter, even more in particular between about 150 mg/liter and about 210 mg/liter.

In accordance with possible forms of embodiment, combinable with all the forms of embodiment described here, the elenolic acid (EA) content can be comprised between about 500 mg/liter and about 1800 mg/liter, in particular between 600 mg/liter and 1800 mg/liter, in particular between 600 mg/liter and 1700 mg/liter, in particular between about 700 mg/liter and about 1600 mg/liter, more in particular between about 800 mg/liter and about 1500 mg/liter, even more in particular between 850 mg/liter and 1400 mg/liter.

In accordance with possible forms of embodiment, combinable with all the forms of embodiment described here, the decoction according to the present description can also include rutin.

In accordance with possible forms of embodiment, combinable with all the forms of embodiment described here, the rutin content can be comprised between about 100 mg/liter and about 600 mg/liter, in particular between 110 mg/liter and 500 mg/liter, in particular between 120 mg/liter and 400 mg/liter, in particular between about 125 mg/liter and about 300 mg/liter, even more in particular between 130 mg/liter and 280 mg/liter.

Forms of embodiment of a decoction in accordance with the present description, or a concentrated compound obtained from the decoction, can be used for the preparation of products comprising the decoction, in particular food or drink products, also dietary supplements, cosmetics, pharmaceuticals, detergents, deodorants, disinfectants, sanitizing or hygiene products, or industrial products, for human use or veterinary/animal use, or for agricultural use, in particular for example as a pesticide, antimicotic and/or antimicrobic in plants. These products are all included within the field of protection of the present invention.

Possible examples of embodiment are food creams or food products in general that contain the decoction in accordance with the present description or a concentrated compound obtained from the decoction.

For example, the decoction, or a concentrated compound obtained from the decoction, can be used in food products that require a reduced quantity of water.

An example of a food product can be a confectionary product containing the decoction or a cream or filling in a creamy form or a dense/viscous liquid, based on a decoction according to the present description or a concentrated compound obtained from the decoction.

A confectionary product usable in association with the forms of embodiment described here can be a chocolate based product.

A specific example can be solid chocolate, in the form of a bar for example, based on or containing the decoction in question or a concentrated compound obtained from the decoction. For example, solid chocolate can be provided, containing a concentrated compound obtained from the decoction according to the present description.

Another specific example can be a chocolate tasting spreadable cream, based on or containing the decoction in question or a concentrated compound obtained from the decoction. For example, a chocolate based spreadable cream can be provided, containing a concentrated compound obtained from the decoction in accordance with the present description.

Another specific example can be a chocolate filled with a creamy composition or dense/viscous liquid, based on or containing the decoction in question or a concentrated compound obtained from the decoction.

Another confectionary product usable in association with the forms of embodiment described here can be a product based on honey.

A specific example can be honey based on or containing the decoction in question or a concentrated compound obtained from the decoction. For example it can be honey containing a concentrated compound obtained from the decoction in accordance with the present description.

Forms of embodiment described here concern a decoction in accordance with the present description for use in therapeutic treatment.

Forms of embodiment described here concern the use of a decoction in accordance with the present description as an antioxidant.

Forms of embodiment described here concern the use of a decoction in accordance with the present description as a stimulant.

Forms of embodiment described here concern the use of a decoction in accordance with the present description as radical scavenger.

Forms of embodiment described here concern the use of a decoction in accordance with the present description or product containing said decoction or based on a concentrated compound obtained from the decoction, in the treatment of tanning hides. In particular, Applicant has found an effective and advantageous effect in tanning hides, thanks to the use of the tannins present in the decoction in accordance with the present description. For example it is possible to use the tannins present in the decoction in accordance with the present description in the tannery sector, for example for furniture, for example covering for furniture as in the domestic sector, or in the automobile, naval or aeronautical sector, or in the leather goods sector, furnishings or furnishing accessories or other.

Forms of embodiment described here concern the use of a decoction in accordance with the present description or product containing said decoction or based on a concentrated compound obtained from the decoction, as a pesticide for vegetables. Advantageously this is due to the antimicotic and antimicrobic properties of the decoction in accordance with the present description.

Forms of embodiment described here concern food supplements comprising the decoction in accordance with the present description or a concentrated compound obtained from the decoction.

Possible implementations of food supplements are food supplements in liquid form, in solid form, in lyophilized powders, in atomized powders or dehydrated powders.

Forms of embodiment described here concern a concentrated compound obtained from a decoction in accordance with the present description. A concentrated compound as understood in the form of embodiment described here can be a concentrated dry compound, in particular a concentrated dry compound in powder form, for example obtained by means of atomization or lyophilization.

In accordance with one form of embodiment, the concentrated compound can provide a concentrate yield (dry yield or dry residue yield) comprised between 1% and 4%, in particular between 1.1% and 2.5%, more in particular between 1.2% and 1.6%, even more in particular between 1.25% and 1.5%, expressed as a percentage ratio of grams of final concentrated compound (also called dry residue) on millimeters of volume of the basic decoction. Examples of concentrate yield can be: 1.25%, 1.30%, 1.35%, 1.40%, 1.43%, 1.45%. Other examples of concentrate yield can be: 2.50%, 3%, 3.5%, 4%.

Forms of embodiment described here concern a viscous concentrated compound, obtained from a decoction in accordance with the present description.

Forms of embodiment described here concern cosmetics comprising the decoction in accordance with the present description or a concentrated compound obtained from the decoction.

Possible implementations of cosmetics are body creams, face creams, anti-ageing cream, compounds for oral hygiene, compounds for personal hygiene, lotions for the hair, shampoos, bubble-baths, aftershave lotions, shaving creams, sun protection creams, tanning lotions, tanning sprays, beauty products (for example lipstick, eyeshadow, blushers . . . ), slimming creams, anti-cellulite creams.

Forms of embodiment described here concern soaps comprising the decoction in accordance with the present description or a concentrated compound obtained from the decoction.

Forms of embodiment described here concern deodorants comprising the decoction in accordance with the present description or a concentrated compound obtained from the decoction.

Forms of embodiment described here concern perfumes comprising the decoction in accordance with the present description or a concentrated compound obtained from the decoction.

Forms of embodiment described here concern depilatories comprising the decoction in accordance with the present description or a concentrated compound obtained from the decoction.

Forms of embodiment described here concern massage oils comprising the decoction in accordance with the present description or a concentrated compound obtained from the decoction.

Forms of embodiment described here concern medicines comprising the decoction in accordance with the present description or a concentrated compound obtained from the decoction.

Possible implementations of medicines are oral preparations, topical preparations (creams, gels, ointments, pastes), nasal preparations, parenteral preparations, pharmaceutical preparations.

Forms of embodiment described here concern foods in general comprising the decoction in accordance with the present description or a concentrated compound obtained from the decoction.

Possible implementations of foods that can be added, mixed or incorporated with the decoction according to the present description or a concentrated compound obtained from the decoction are for example baked products and/or doughs to obtain the baked products, pasta, proteinous pasta, creams, sauces and juices, marmalades and jams, fruit gelatins, and food preserves in general, soups, mousses, condiments, food oils, in particular olive oil, more in particular extra virgin olive oil, or oils made from fruit (for example nuts, currants, almonds, hazel nuts, apricot, peach, avocado, cherry, water melon, melon, blueberry, coconut, grape), oils from cereals (for example corn, rice, wheat germ, barley, oats, rye, sorghum, millet), or frozen products, doughs, cakes, ice-creams and sorbets. Aromas, spices, aromatic herbs, vegetable extracts, preservatives, acidifiers, antioxidants and emulsifying agents can also be optionally added.

Forms of embodiment described here concern non-alcoholic drinks comprising the decoction in accordance with the present description or a concentrated compound obtained from the decoction. For example the non-alcoholic drinks can include a drink made with water, non-fermented juices, for example based on grapes or other fruit (pear, peach, pineapple, apricot, banana, orange, blueberry, strawberry or other fruits), or non-alcoholic drinks, such as fruit juices or traditional drinks of the cola type, either carbonated or non-carbonated. The chosen fruit juice or non-alcoholic drink can be diluted with water, or not, and can be a mixture of several fruit juices, with or without added sugar or other sweetening ingredients, carbonated or not. Or other non-alcoholic drinks can be provided, not necessarily deriving from fruit or fruit juices, carbonated or not.

Other non-alcoholic drinks can also be provided, such as energy drinks, non-alcoholic aperitifs, carbonated or non-carbonated drinks in general.

Forms of embodiment described here concern alcoholic drinks comprising the decoction in accordance with the present description or a concentrated compound obtained from the decoction. For example the alcoholic drinks can include an alcoholic liquid based on grapes. By alcoholic liquid based on grapes we mean an alcoholic liquid deriving at least from the fermentation of grape juice, which can be for example red wine, white wine or a distillate. For example the alcoholic content of the alcoholic liquid based on grapes can be comprised between 4 and 50 percent proof. In practice the range goes from grape wine to distillates. For example, in the case of wine an alcoholic liquid is comprised between 9 and 15 percent proof. In another example, the alcoholic drink can comprise an alcoholic liquid based on cereals, that is, deriving at least from the fermentation of cereals. It is also possible to use an alcoholic drink that includes an alcoholic liquid based on sugar cane, that is, deriving at least from the fermentation of sugar cane. It is also possible to use an alcoholic drink that includes an alcoholic liquid based on tubers, such as potatoes, that is, deriving at least from the fermentation of tubers. In general, it is possible to use an alcoholic drink that can include an alcoholic liquid based on a vegetable, that is, deriving from the fermentation of a vegetable.

The decoction or concentrated compound obtained from the decoction can also be in itself subjected to a fermentation process, when it is suitably mixed with juices and/or fruit and vegetables containing sugar, as well as honey, so as to obtain, from the fermentation of these mixtures, alcoholic drinks, also sparkling, dry, demi-sec or sweet.

Forms of embodiment described here concern energy drinks or energizers comprising the decoction in accordance with the present description or a concentrated compound obtained from the decoction.

Forms of embodiment described here concern domestic detergents comprising the decoction in accordance with the present description or a concentrated compound obtained from the decoction.

Forms of embodiment described here concern sanitizers for rooms or for sanitary accessories comprising the decoction in accordance with the present description or a concentrated compound obtained from the decoction.

Forms of embodiment described here concern deodorants for rooms or for sanitary accessories comprising the decoction in accordance with the present description or a concentrated compound obtained from the decoction.

Forms of embodiment described here concern hygiene products comprising the decoction in accordance with the present description or a concentrated compound obtained from the decoction.

Forms of embodiment described here concern industrial detergents comprising the decoction in accordance with the present description or a concentrated compound obtained from the decoction.

Forms of embodiment described here concern clinical detergents comprising the decoction in accordance with the present description or a concentrated compound obtained from the decoction.

Forms of embodiment described here concern products for tanning hides comprising the decoction in accordance with the present description or a concentrated compound obtained from the decoction.

Forms of embodiment described here concern products for animals comprising the decoction in accordance with the present description or a concentrated compound obtained from the decoction.

Possible implementations of products for animals are fodder, supplementary fodder, animal food supplements, animal detergents, anti-parasitic products, drinks for animals and veterinary medicines.

Active or auxiliary substances can be added to the possible forms of embodiments and implementations described here. Said active or auxiliary substances are selected from a group comprising: vitamins, including vitamin A (retinol), vitamins from the B group, including vitamins B1, B2, B3 (PP, niacin), B5, B6, B12, vitamin C (ascorbic acid), vitamin D, vitamin E (tocopherol), vitamin H, minerals or metals, such as magnesium, potassium, zinc, selenium, calcium, fluoride, phosphorous, complementary nutrients such as creatine, carnitine, gamma oryzanol, lipoic acid, choline, carnosine, phosphatidylserine, lutein, lycopene, resveratrol, other antioxidants (such as for example coenzyme Q10, lipoic acid, tocotrienol, glutathione, flavonoids, other polyphenols, as well as the retinol, ascorbic acid, tocopherols, lycopene and resveratrol already cited), essential fatty acids omega-3, for example EPA and/or DHA, and omega-6, other vegetable substances, extracts and/or preparations other than those from the olive tree, such as for example extract of Guarana, Blueberry, Ginseng, Ginkgo biloba, Rhodiola rosea, Boswellia serrata, yeast, serum proteins or proteins from milk, intense sweeteners such as xylitol, erythritol, mannitol, sorbitol, amino acids, sulphurated amino acids, branched-chain or modified amino acids or other active or auxiliary substances.

For example, forms of embodiment can provide possible compositions containing the decoction according to the present description or a concentrated compound obtained from the decoction that can be made and formulated in products suitable to be used for preventative and curative purposes such as medicines or food supplements or cosmetic products. For these uses the compositions in accordance with forms of embodiment can be made in liquid or powder form, even in sachets of one dose, or in a mixture with excipients and diluents acceptable from the pharmaceutical or para-pharmaceutical, diet-food point of view. They can also be used in different forms, such as capsules, tablets, pastes, gels, solutions or suspensions, sprays with excipients and diluents acceptable from the pharmaceutical or para-pharmaceutical, diet-food point of view, and suitable for these other forms. Moreover, for cosmetic para-pharmaceutical uses the compositions in accordance with some forms of embodiment can be formulated with excipients and diluents acceptable from the cosmetic point of view in the form of lotions, creams, salves, pastes, gels, plasters, mousses, foams, sticks and sprays and other topical forms known for this use.

The present invention also concerns forms of embodiment obtained from the decoction according to the present description using chemical, mechanical, physical, chemical-physical and/or thermodynamic treatment, including for example thermal variation techniques, that is, the increase or reduction in temperature, and/or pressure treatment, that is the increase or reduction in pressure, for example precipitation, freezing, evaporation, atomization, lyophilization, granulation, spraying, pelletization, coalescence, gelation, grinding, crushing, pulverization, filtration or other. These forms of embodiment can be used to obtain a concentrated compound starting from a decoction according to the present description.

### Examples

One example of a possible implementation of a decoction according to the present description provides:

oleuropein (OE) from about 2100 mg/liter to about 2300 mg/liter;

hydroxytyrosol (HT) from about 130 mg/liter to about 165 mg/liter;

tyrosol (T) from about 115 mg/liter to about 140 mg/liter;

elenolic acid (EA) from about 1150 mg/liter to about 1250 mg/liter.

Another example of a possible implementation of a decoction according to the present description provides:

oleuropein (OE) from about 2150 mg/liter to about 2250 mg/liter;

hydroxytyrosol (HT) from about 135 mg/liter to about 160 mg/liter;

tyrosol (T) from about 120 mg/liter to about 130 mg/liter;

elenolic acid (EA) from about 1190 mg/liter to about 1230 mg/liter.

Another example of a possible implementation of a decoction according to the present description provides:

oleuropein (OE) from about 2180 mg/liter to about 2220 mg/liter;

hydroxytyrosol (HT) from about 145 mg/liter to about 155 mg/liter;

tyrosol (T) from about 125 mg/liter to about 128 mg/liter;

elenolic acid (EA) from about 1210 mg/liter to about 1220 mg/liter.

Another example of a possible implementation of a decoction according to the present description provides:

oleuropein (OE) from about 1600 mg/liter to about 1700 mg/liter;

hydroxytyrosol (HT) from about 105 mg/liter to about 120 mg/liter;

tyrosol (T) from about 90 mg/liter to about 100 mg/liter;

elenolic acid (EA) from about 850 mg/liter to about 920 mg/liter.

Another example of a possible implementation of a decoction according to the present description provides:

oleuropein (OE) from about 1650 mg/liter to about 1680 mg/liter;

hydroxytyrosol (HT) from about 108 mg/liter to about 118 mg/liter;

tyrosol (T) from about 92 mg/liter to about 98 mg/liter;

elenolic acid (EA) from about 875 mg/liter to about 900 mg/liter.

Another example of a possible implementation of a decoction according to the present description provides:

oleuropein (OE) from about 1660 mg/liter to about 1670 mg/liter;

hydroxytyrosol (HT) from about 110 mg/liter to about 115 mg/liter;

tyrosol (T) from about 94 mg/liter to about 97 mg/liter;

elenolic acid (EA) from about 880 mg/liter to about 890 mg/liter.

Another example of a possible implementation of a decoction according to the present description provides:

oleuropein (OE) from about 2500 mg/liter to about 2700 mg/liter;

hydroxytyrosol (HT) from about 200 mg/liter to about 220 mg/liter;

tyrosol (T) from about 165 mg/liter to about 190 mg/liter;

elenolic acid (EA) from about 1350 mg/liter to about 1500 mg/liter.

Another example of a possible implementation of a decoction according to the present description provides:

oleuropein (OE) from about 2600 mg/liter to about 2670 mg/liter;

hydroxytyrosol (HT) from about 205 mg/liter to about 218 mg/liter;

tyrosol (T) from about 170 mg/liter to about 180 mg/liter;

elenolic acid (EA) from about 1370 mg/liter to about 1450 mg/liter.

Another example of a possible implementation of a decoction according to the present description provides:

oleuropein (OE) from about 2650 mg/liter to about 2660 mg/liter;

hydroxytyrosol (HT) from about 210 mg/liter to about 215 mg/liter;

tyrosol (T) from about 172 mg/liter to about 176 mg/liter;

elenolic acid (EA) from about 1390 mg/liter to about 1410 mg/liter.

Another example of a possible implementation of a decoction according to the present description provides:

oleuropein (OE) from about 500 mg/liter to about 600 mg/liter;

hydroxytyrosol (HT) from about 130 mg/liter to about 165 mg/liter;

tyrosol (T) from about 115 mg/liter to about 140 mg/liter;

elenolic acid (EA) from about 400 mg/liter to about 500 mg/liter.

Another example of a possible implementation of a decoction according to the present description provides:

oleuropein (OE) from about 550 mg/liter to about 650 mg/liter;

hydroxytyrosol (HT) from about 135 mg/liter to about 160 mg/liter;

tyrosol (T) from about 120 mg/liter to about 130 mg/liter;

elenolic acid (EA) from about 450 mg/liter to about 500 mg/liter.

Another example of a possible implementation of a decoction according to the present description provides:

oleuropein (OE) from about 600 mg/liter to about 700 mg/liter;

hydroxytyrosol (HT) from about 145 mg/liter to about 155 mg/liter;

tyrosol (T) from about 125 mg/liter to about 128 mg/liter;

elenolic acid (EA) from about 500 mg/liter to about 600 mg/liter.

Another example of a possible implementation of a decoction according to the present description provides:

oleuropein (OE) from about 650 mg/liter to about 750 mg/liter;

hydroxytyrosol (HT) from about 105 mg/liter to about 120 mg/liter;

tyrosol (T) from about 90 mg/liter to about 100 mg/liter;

elenolic acid (EA) from about 550 mg/liter to about 650 mg/liter.

Another example of a possible implementation of a decoction according to the present description provides:

oleuropein (OE) from about 750 mg/liter to about 850 mg/liter;

hydroxytyrosol (HT) from about 108 mg/liter to about 118 mg/liter;

tyrosol (T) from about 92 mg/liter to about 98 mg/liter;

elenolic acid (EA) from about 600 mg/liter to about 700 mg/liter.

Another example of a possible implementation of a decoction according to the present description provides:

oleuropein (OE) from about 800 mg/liter to about 900 mg/liter;

hydroxytyrosol (HT) from about 110 mg/liter to about 115 mg/liter;

tyrosol (T) from about 94 mg/liter to about 97 mg/liter;

elenolic acid (EA) from about 650 mg/liter to about 750 mg/liter.

Another example of a possible implementation of a decoction according to the present description provides:

oleuropein (OE) from about 850 mg/liter to about 950 mg/liter;

hydroxytyrosol (HT) from about 200 mg/liter to about 220 mg/liter;

tyrosol (T) from about 165 mg/liter to about 190 mg/liter;

elenolic acid (EA) from about 700 mg/liter to about 800 mg/liter.

Another example of a possible implementation of a decoction according to the present description provides:

oleuropein (OE) from about 900 mg/liter to about 1000 mg/liter;

hydroxytyrosol (HT) from about 205 mg/liter to about 218 mg/liter;

tyrosol (T) from about 170 mg/liter to about 180 mg/liter;

elenolic acid (EA) from about 750 mg/liter to about 850 mg/liter.

Another example of a possible implementation of a decoction according to the present description provides:

oleuropein (OE) from about 950 mg/liter to about 1050 mg/liter;

hydroxytyrosol (HT) from about 210 mg/liter to about 215 mg/liter;

tyrosol (T) from about 172 mg/liter to about 176 mg/liter;

elenolic acid (EA) from about 800 mg/liter to about 900 mg/liter.

### Experimental Data

The Applicant conducted analyses to evaluate the concentration of some chemical constituents in decoctions of olive leaves in accordance with the present description, in order to measure the antioxidant and anti-radical power in vitro and to evaluate the stimulating activity in vivo. In particular, using high-performance liquid chromatography (HPLC) and liquid chromatography coupled with mass spectrometry (LC-MS) the concentration of oleuropein, hydroxytyrosol, tyrosol, elenolic acid and rutin were evaluated in three different decoctions of olive leaves (1-3) according to the present description.

The antioxidant and anti-radical activity was studied on decoctions 1 and 2, respectively, by testing the decolorization of the radical cation ABTS^{•} (2.2′-azino-bis-3-ethylbenzothiazoline-6-sulphonic acid) (Re et al.—Free Rad. Biol. Med. 26: 1231, 1999) and by testing the neutralization of the radical 1,1-diphenyl-2-picrylhydrazyl (DPPH^{•}) (Brand-Williams et al.—Food Sci. Technol. 28: 25, 1995). Finally, decoctions 1 and 2 were studied for potential stimulating activity, verifying the effect on sleep induced by ethanol in mice according to the method reported by Yacoubi et al. (Yacoubi et al. —Neuropharmacology 45: 977, 2003).

### Materials and Methods

### Materials and Animals

The decoctions of olive leaves in accordance with the present description analyzed were the following: decoction 1, decoction 2, decoction 3.

These were lyophilized before carrying out the tests. Alternatively, the decoctions could be subjected to atomization.

The green tea leaves were bought at a health food shop. Oleuropein, hydroxytyrosol, tyrosol, rutin, caffeine, ABTS^{•}, DPPH^{•}, Trolox and the other reagents and solvents were bought from Sigma Aldrich (Milan, Italy). The elenolic acid was obtained by means of enzymatic hydrolysis starting from oleuropein. The animals used were mice CD-1 males (average weight 28-32 g) bought from Harlan Laboratories (S. Pietro al Natisone, UD, Italy).

### Quantification of Oleuropein, Hydroxytyrosol, Tyrosol, Elenolic Acid and Rutin.

The quantity of oleuropein, hydroxytyrosol, tyrosol, and rutin in the decoction of olive leaves was determined by means of high-performance liquid chromatography (HPLC). The lyophilized decoctions were dissolved in ethanol, filtered with a Millipore filter (0.45 μm) and subjected to HLPC analysis as reported by Benavente-Garcia and collaborators (Benavente-Garcia et al.—Food Chem. 68: 457, 2000). The elenolic acid was determined by means of liquid chromatography coupled with mass spectrometry (LC-MS) according to the method reported by Bazoti and collaborators (Bazoti et al.—Biomol. Chromatogr. 24: 506, 2010). For each sample four analyses were carried out and the result was expressed as average±standard deviation.

### Evaluation of the Antioxidant Activity

The antioxidant activity was evaluated according to the method reported by Re and collaborators (Re et al.—Free Rad. Biol. Med. 26: 1231, 1999). This test was based on the use of monocation radical form of 2,2′-azino-bis-3-ethylbenzothiazoline-sulphonic acid (ABTS^{•+}), blue in color. In the presence of an antioxidant the ABTS^{•+} is reduced to colorless ABTS, determining a decoloring of the solution in which it is dissolved, which can be measured by spectrophotometry quantifying the absorbance at 734 nm. The antioxidant activity of the samples under examination was measured at different concentrations and, from the straight lines that put the concentrations analyzed in relation to the percentages of reduction of absorbance registered, for each sample the value of concentration able to reduce the absorbance by 50% (IC50) was calculated. The antioxidant activity, expressed as IC50, was compared to that of Trolox, a similar water-soluble to vitamin E, and to that of an infusion of green tea, prepared with 10 g of leaves in 1 liter of water (from which 4.385 g of lyophilized infusion were obtained). For every sample four determinations were carried out and the result was expressed as average±standard deviation of the analyses done.

### Evaluation of the Radical Scavenger Activity

The radical scavenger activity was evaluated according to the method reported by Brand-Williams and collaborators (Brand-Williams et al.—Food Sci. Technol. 28: 25, 1995). The test is based on the use of the stable radical 1,1-diphenyl-2-picrylhydrazyl (DPPH^{•}), violet in color. In the presence of a radical scavenger the radical DPPH^{•} is reduced to a yellow colored compound. This reaction can be monitored by spectrophotometry, measuring the reduction in absorbance of the solution at 517 nm. The radical scavenger activity of the samples under examination was measured at different concentrations and, from the straight lines that put the concentrations analyzed in relation to the percentages of reduction of the absorbance registered, for every sample the value of concentration able to reduce absorbance by 50% (IC50) was calculated. The radical scavenger activity, expressed as IC50, was compared to that of an infusion of green tea, obtained as reported in the previous paragraph. For every sample four determinations were carried out and the result was expressed as average±standard deviation of the analyses done.

### Evaluation of the Stimulant Effect

The stimulating activity of the decoctions of olive leaves was evaluated on the basis of the reduction of the hypnotic effect induced by ethanol in the mice (Yacoubi et al.—Neuropharmacology 45: 977, 2003). The animals were housed in groups of 5 and kept for at least a week in the same place where the test was carried out, at a constant temperature (23±1° C.) and relative humidity (50-60%), with a fixed artificial light cycle (07.00-19.00). The experiment was carried out in conformity with the laws in force that regulate the use of animals for scientific purposes (Italian Legislative Decree 116 of 27 Jan. 1992 and the Directives of the European Communities Council of 24 Nov. 1986/86/609/EEC).

At the moment of the experiment, each animal was positioned individually in a cage. The lyophilized decoctions, dissolved in water, were administered to the mice (dose: 120 mg/kg; volume of solution administered: 10 ml/kg) by means of gastric intubation, 30 minutes before the inter-peritoneal injection of an aqueous composition of ethanol at 20% (v/v; 4 g/kg). The control animals were administered 10 ml/kg of physiological solution. The induction of ethanol induced sedation in the animals, with the loss of the straightening reflex. In every animal both the duration of the latency phase that elapsed between the injection of the ethanol and the loss of the straightening reflex, and the duration of the loss of the straightening reflex were measured. The stimulating activity was evaluated on the basis of both the increase in the latency phase and also on the reduction in the duration of the loss of the straightening reflex. The effect was compared to that of caffeine, at a dose of 60 mg/kg. Each treatment group consisted of 10 animals.

### Statistical Analysis

The values of IC50 were calculated on the basis of the linear regression of the straight lines that put the logarithm of the concentrations tested in relation to the reduction of the absorbance measured, using the appropriate function of an electronic spreadsheet. The in vivo data were analyzed according to the Student's t-test, accepting as significant the values of p<0.05.

### Results

### Lyophilization Yield of the Decoctions

The decoctions of olive leaves were subjected to lyophilization before being subjected to phytochemical analyses, antioxidant activity tests and evaluation of the stimulant effect. The lyophilization yields, shown in Table 1, are 1.25% (decoction 1), 1.01% (decoction 2) and 1.43% (decoction 3).

TABLE 1
Lyophilization yields of decoctions

| Decoction | Lyophilized volume (ml) | Yield (g) | Yield (%) |
|---|---|---|---|
| 1 | 200 | 2.50 | 1.25 |
| 2 | 200 | 2.02 | 1.01 |
| 3 | 200 | 2.86 | 1.43 |

### Quantification of Oleuropein, Hydroxytyrosol, Tyrosol, Elenolic Acid and Rutin

The results of the analysis of decoctions 1, 2 and 3, intended to quantify the oleuropein, hydroxytyrosol, tyrosol, elenolic acid and rutin, are shown in Table 2.

TABLE 2
Concentration of hydroxytyrosol, tyrosol, elenotic
acid and rutin in decoctions of olives leaves

| | Decoction 1 | | Decoction 2 | | Decoction 3 | |
|---|---|---|---|---|---|---|
| | Lyophilized | | Lyophilized | | Lyophilized | |
| | (mg/g) | | (mg/g) | | (mg/g) | |
| | [Decoction | | [Decoction | | [Decoction | |
| Compound | (mg/l)] | | (mg/l)] | | (mg/l)] | |
| Oleuropein | 176.0 | [2200] | 165.1 | [1668] | 185.7 | [2656] |
| Hydroxytyrosol | 12.1 | [151] | 11.3 | [114] | 14.9 | [213] |
| Tyrosol | 10.2 | [127] | 9.5 | [96] | 12.2 | [174] |
| Elenolic acid | 97.2 | [1215] | 87.3 | [882] | 97.4 | [1393] |
| Rutin | 14.7 | [184] | 13.6 | [137] | 16.6 | [237] |

For each sample, the concentration of the compounds was expressed both in terms of milligrams per gram of lyophilized decoction, and also in terms of milligrams per liter of liquid decoction. From the analysis, it emerged that, in all three decoctions, oleuropein is the most abundant compound (165.1-185.7 mg/g of lyophilized decoction, corresponding to 1668-2656 mg/l of liquid decoction), followed by elenolic acid (87.3-97.4 mg/g of lyophilized decoction, corresponding to 882-1393 mg/l of liquid decoction), by rutin (13.6-16.6 mg/g of lyophilized decoction, corresponding to 137-237 mg/l of liquid decoction), by hydroxytyrosol (12.1-14.9 mg/g of lyophilized decoction, corresponding to 114-213 mg/l of liquid decoction) and by tyrosol (9.5-12.2 mg/g of lyophilized decoction, corresponding to 96-174 mg/l of liquid decoction). Moreover, it emerged that the beneficial bioactive compounds analyzed are more abundant in decoction 3.

### Antioxidant Activity of the Decoctions

The results of the antioxidant activity of the samples under examination, evaluated by means of the decoloring test of the cationic radical ABTS^{•+}, are shown in Table 3. The antioxidant activity of each sample was evaluated at different concentrations and was expressed as the concentration of a substance able to reduce the absorbance of the solution under examination by 50% (IC50), with respect to a control solution, without the samples under examination.

The three decoctions showed a similar antioxidant activity with respect to each other: the IC50 values expressed as milligrams of lyophilized decoction per millimeter are comprised between 0.25 and 0.32 mg/ml.

Expressing the values of IC50 as microliters of liquid decoction per millimeter of solution analyzed, the IC50 is comprised between 18.9 and 25.6 μg/ml and the antioxidant activity of decoction 3 (IC50=18.9 μg/ml) seems slightly bigger than that of decoctions 1 and 2 (IC50=25.6 and 24.8 μg/ml, respectively). As expected, the IC50 of Trolox, used as a reference, was equal to 0.03 mg/ml, while that of the infusion of green tea was equal to 0.25 mg lyophilized infusion/ml, corresponding to 57.0 μl liquid infusion/ml (Table 3).

TABLE 3
Antioxidant activity of the decoctions of olive leaves

| | Lyophilized Decoction | Liquid Decoction |
|---|---|---|
| | IC₅₀ (mg/ml) | IC₅₀ (μl/ml) |
| Preparation | Average ± D.S. | Average ± D.S. |
| Decoction 1 | 0.32 ± 0.04 | 25.6 ± 2.9 |
| Decoction 2 | 0.25 ± 0.03 | 24.8 ± 2.5 |
| Decoction 3 | 0.27 ± 0.03 | 18.9 ± 2.3 |
| Trolox | 0.03 ± 0.01 | — |
| Green tea | 0.25 ± 0.03 | 57.0 ± 6.2 |

### Radical Scavenger Activity of the Decoctions

The result of the radical scavenger activity of the samples under examination, evaluated using the color variation test of the DPPH^{•} radical, are shown in Table 4. The antiradical activity of each sample, evaluated at different concentrations, was expressed as the concentration of a substance able to reduce the absorbance of the solution under examination by 50% (IC50), with respect to a control solution, without the samples under examination.

The three decoctions of olive leaves have shown radical scavenger properties to the neutralization test of the DPPH^{•} radical, with values of IC50 comprised between 0.24 and 0.38 mg of lyophilized decoction per millimeter, corresponding to 23.8 and 26.6 μl liquid decoction/ml. The antiradical activity of decoction 2 seems slightly bigger than that of the other two. As reference, the IC50 of the infusion of green tea was equal to 0.14 mg of lyophilized infusion/ml, corresponding to 31.9 μl liquid infusion/ml (Table 4).

TABLE 4
Radical scavenger activity of the decoction of olive leaves

| | Lyophilized Decoction | Liquid Decoction |
|---|---|---|
| | IC₅₀ (mg/ml) | IC₅₀ (μl/ml) |
| Preparation | Average ± D.S. | Average ± D.S. |
| Decoction 1 | 0.32 ± 0.04 | 25.6 ± 2.8 |
| Decoction 2 | 0.24 ± 0.04 | 23.8 ± 2.6 |
| Decoction 3 | 0.38 ± 0.05 | 26.6 ± 2.9 |
| Green tea | 0.14 ± 0.03 | 31.9 ± 3.6 |

### Stimulating Activity in Mice

The stimulating activity of decoctions 1 and 2 was evaluated in the mice, after oral administration of 120 mg/kg of lyophilized decoction (corresponding to 9.6 and 11.9) ml of decoction 1 and 2 liquid/kg), verifying the effect on the loss of straightening induced by the intraperitoneal injection of ethanol (4 g/kg). The parameters evaluated were the increase in the latency phase between the ethanol injection and the loss of the straightening reflex, and the duration of the loss of the straightening reflex (interval between the loss and the re-acquisition of the straightening reflex). As a reference compound, caffeine was used at a dose of 60 mg/kg (corresponding to an average dose of coffee equal to about 40 ml/kg).

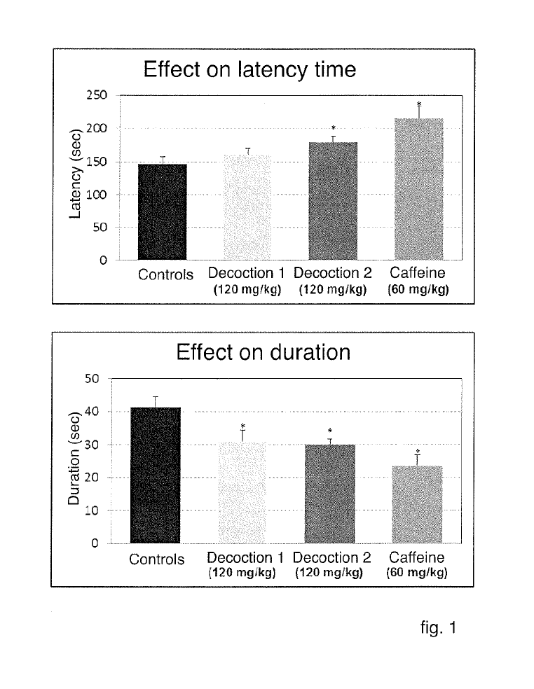

The results obtained are reported in Table 5 and shown graphically in FIG. 1, which describes the effect of the decoctions and the caffeine on the latency between the ethanol injection and the loss of the straightening reflex and on the duration of the latter (*p<0.05 according to the Student's t-test, with respect to the controls).

Considering the latency between the ethanol injection and the loss of the straightening reflex, only decoction 2 and the caffeine were able to determine a statistically significant increase in the latency period, equal to 23% and 48%, respectively. On the contrary, both decoction 1 and decoction 2 were able to determine a significant reduction in the duration of the loss of the straightening reflex, equal to 25% and 28%. As a reference, caffeine was able to reduce the duration by 43% (Table 5).

TABLE 5
Stimulating activity of decoctions of olive leaves: effect on latency
time and duration of the loss of straightening reflex in mices

| | | | Differ- | | Differ- |
|---|---|---|---|---|---|
| Treatment | Dose | Latency (sec) | ence | Duration (min) | ence |
| group | (mg/kg) | Average ± E.S. | (%) | Average ± E.S. | (%) |
| Controls | — | 145.5 ± 11.7 | — | 41.4 ± 3.2 | — |
| Decoction | 120 | 161.9 ± 8.1 | 11 | 31.2 ± 3.2* | −25 |
| 1 | | | | | |
| Decoction | 120 | 179.5 ± 9.2* | 23 | 29.9 ± 1.8* | −28 |
| 2 | | | | | |
| Caffeine | 60 | 215.7 ± 18.2* | 48 | 23.5 ± 3.3* | −43 |

*p < 0.05 according to the Student's t-test, with respect to the controls; n = 10 animals for group

It must be stressed that the same results as above can be obtained using, instead of the lyophilization technique, the technique of atomizing the decoction, which essentially provides an extraction of the water from the decoction, under conditions of reduced pressure, therefore operating at low temperature.

### EXPERIMENTAL CONCLUSIONS

The preliminary results of the experimental tests carried out by Applicant have shown that:

(1) the three decoctions of olive leaves analyzed in the experimental tests have a oleuropein content of 1668-2656 mg/l, hydroxytyrosol of 114-213 mg/l, tyrosol of 96-174 mg/l, elenolic acid of 882-1393 mg/l and rutin of 137-237 mg/l; these beneficial bioactive compounds were more abundant in decoction 3;
(2) decoctions 1 and 2 have an antioxidant activity and radical scavenger activity greater than that of the infusion of green tea;
(3) decoctions 1 and 2 have stimulating properties, shown in vivo on the basis of the ability to increase the duration of the loss of the straightening reflex in mice, induced by ethanol;
(4) the conclusions of points (2) and (3) can be extended, hypothesizing an even greater effect, to decoction 3 by virtue of the fact that the cited beneficial bioactive compounds, in particular OE, HT, T and EA and optionally also rutin, were more abundant in decoction 3.

It is clear that modifications and/or additions of parts may be made to the decoction of olive leaves as described heretofore, without departing from the field and scope of the present invention.

It is also clear that, although the present invention has been described with reference to some specific examples, a person of skill in the art shall certainly be able to achieve many other equivalent forms of decoction of olive leaves, having the characteristics as set forth in the claims and hence all coming within the field of protection defined thereby.

1. Decoction of olive leaves comprising an aqueous composition containing at least:
from about 500 mg/liter to about 3,000 mg/liter of oleuropein;
from about 100 mg/liter to about 300 mg/liter of hydroxytyrosol;
from about 90 mg/liter to about 280 mg/liter of tyrosol;
from about 400 mg/liter to about 1,800 mg/liter of elenolic acid;
from about 100 mg/liter to about 600 mg/liter of rutin;
wherein said decoction is an extract of said olive leaves obtained by hot water extraction.

2. Decoction as in claim 1, wherein said decoction has a slightly bitter taste.

3. Decoction as in claim 1, wherein the content of oleuropein is comprised between about 600 mg/liter and about 2,850 mg/liter.

4. Decoction as in claim 1, wherein the content of oleuropein is comprised between about 700 mg/liter and about 2,800 mg/liter.

5. Decoction as in claim 1, wherein the content of hydoxytyrosol is comprised between about 110 mg/liter and about 290 mg/liter.

6. Decoction as in claim 1, wherein the content of hydoxytyrosol is comprised between about 140 mg/liter and about 280 mg/liter.

7. Decoction as in claim 1, wherein the content of tyrosol is comprised between about 95 mg/liter and about 270 mg/liter.

8. Decoction as in claim 1, wherein the content of tyrosol is comprised between about 110 mg/liter and about 250 mg/liter.

9. Decoction as in claim 1, wherein the content of elenolic acid is comprised between about 600 mg/liter and about 1,800 mg/liter.

10. Decoction as in claim 1, wherein the content of elenolic acid is comprised between about 600 mg/liter and about 1,700 mg/liter.

11. Decoction as in claim 1, wherein the content of elenolic acid is comprised between about 700 mg/liter and about 1,600 mg/liter.

12. Product comprising a decoction as in claim 1, said product being chosen from a group comprising: food or drink products, including products of diet integration, cosmetics, pharmaceuticals, detergents, deodorants, disinfectants, sanitizing, hygiene or industrial products, for human use or veterinary/animal use, or agricultural use, in particular as an anti-parasitic, anti-micotic and/or anti-microbe in plants.

13. Concentrated compound obtained from a decoction as in claim 1.

14. Concentrated compound as in claim 13, wherein the concentrate yield is comprised between 1% and 4%, in particular between 1.1% and 2.5%, more in particular between 1.2% and 1.6%, even more in particular between 1.25% and 1.5%, expressed as a percentage ratio of grams of final concentrated compound on millimeters of decoction volume.

15. Viscous concentrated compound, obtained from a decoction as in claim 1.

16. Decoction as in claim 1, or product containing said decoction, for use in therapeutic treatment.

17. Use of a decoction as in claim 1 or product containing said decoction, for use as an antioxidant.

18. Use of a decoction as in claim 1, or product containing said decoction, for use as a stimulant.

19. Use of a decoction as in claim 1, or product containing said decoction, for use as a radical scavenger.

20. Use of a decoction as in claim 1, or product containing said decoction, for use in the treatment of tanning hides.

21. Use of a decoction as in claim 1, or product containing said decoction, for use as an anti-parasitic for vegetables.
